(19) United States
(12) Patent Application Publication
(10) Pub. No.: US 2026/0060738 A1
(43) Pub. Date: Mar. 5, 2026

(54) CLOSED-LOOP CRYOABLATION SYSTEM AND METHOD

(71) Applicant: Boston Scientific Scimed, Inc., Maple Grove, MN (US)

(72) Inventors: Nir Tzabar, Lavon (IL); Winston Tan, Plymouth, MN (US); Andrew Kevin Zachman, St. Michael, MN (US); Maciej Wojciech Misiak, Eden Prairie, MN (US); Abigail Blanchette Teschendorf, Blaine, MN (US); Justin Robert Alt, Minneapolis, MN (US)

(21) Appl. No.: 19/304,945

(22) Filed: Aug. 20, 2025

Related U.S. Application Data
(60) Provisional application No. 63/690,187, filed on Sep. 3, 2024.

(51) Int. Cl. A61B 18/02; A61B 18/00

(52) U.S. Cl. CPC A61B 18/02; A61B 2018/00023; A61B 2018/00577; A61B 2018/00595; A61B 2018/0212; A61B 2018/0256; A61B 2018/0262

(57) ABSTRACT

Embodiments herein relate to cryoablation systems. In an embodiment, a cryosurgery system includes a closed-loop working fluid circuit configured to contain a working fluid. The system can include a compressor configured to increase the pressure of the working fluid. The system can include a heat exchanger configured to lower the temperature of the working fluid. The system can include a cryoablation probe having a shaft having a supply tube and a return tube surrounding the supply tube. The system is configured so that, after exiting the first heat exchanger, the working fluid will enter the cryoablation probe, travel to the expansion chamber, expand in the expansion chamber at a Joule-Thomson orifice, travel to the cryoablation probe interface via the return tube, exit the cryoablation probe via the working fluid outlet, enter the compressor, and enter the first heat exchanger.

This application claims the benefit of U.S. Provisional Application No. 63/690,187 filed Sep. 3, 2024, the content of which is herein incorporated by reference in its entirety.

### TECHNICAL FIELD

Embodiments herein relate to cryoablation systems and more particularly to closed-loop systems for cryosurgery.

### BACKGROUND OF THE INVENTION

During cryosurgery, a surgeon may deploy one or more cryoprobes to ablate a target area of a patient anatomy by freezing and thawing the tissue. In one example, a cryoablation probe uses the Joule-Thomson effect to produce cooling or heating of the probe tip. In such cases, the expansion of a cryofluid in the cryoablation probe from a higher pressure to a lower pressure (without heat exchange to the environment) leads to cooling of the device tip. Heat transfer between the expanded cryofluid and the outer walls of the cryoprobe leads to formation of an ice ball in the tissue around the tip and consequent cryoablation of the tissue.

### SUMMARY OF THE INVENTION

In a first aspect, a closed-loop system for cryosurgery, can be included having a closed-loop working fluid circuit configured to contain a working fluid. The system can include a compressor configured to increase a pressure of the working fluid. The system can include a first heat exchanger downstream from the compressor, wherein the first heat exchanger can be configured to lower a temperature of the working fluid. The system can include a cryoablation probe having a shaft, where the shaft includes a supply tube, a return tube surrounding the supply tube, an insulated zone, and an expansion chamber extending distally from the insulated zone. The system can include a cryoablation probe interface. The cryoablation probe interface can include a working fluid inlet and working fluid outlet. The system can be configured so that, after exiting the first heat exchanger, the working fluid will enter the cryoablation probe via the working fluid inlet, travel to the expansion chamber via the supply tube, expand in the expansion chamber at a Joule-Thomson orifice, wherein upon expanding at the Joule Thompson orifice, the working fluid cools to a temperature of less than 150 Kelvin, travel to the cryoablation probe interface via the return tube, exit the cryoablation probe via the working fluid outlet, enter the compressor downstream from the working fluid outlet, and enter the first heat exchanger downstream from the compressor.

In a second aspect, in addition to one or more of the preceding or following aspects, or in the alternative to some aspects, the working fluid includes a mixed refrigerant.

In a third aspect, in addition to one or more of the preceding or following aspects, or in the alternative to some aspects, the mixed refrigerant consists of non-flammable and non-toxic components.

In a fourth aspect, in addition to one or more of the preceding or following aspects, or in the alternative to some aspects, the mixed refrigerant includes at least one of krypton and argon.

In a fifth aspect, in addition to one or more of the preceding or following aspects, or in the alternative to some aspects, the working fluid includes a zeotropic mixture.

In a sixth aspect, in addition to one or more of the preceding or following aspects, or in the alternative to some aspects, the system can further include an accumulator configured to store at least a portion of the working fluid.

In a seventh aspect, in addition to one or more of the preceding or following aspects, or in the alternative to some aspects, the accumulator can be configured to maintain a working pressure of the working fluid.

In an eighth aspect, in addition to one or more of the preceding or following aspects, or in the alternative to some aspects, the system can further include a vacuum, wherein the vacuum can be configured to pull the working fluid out from the cryoablation probe.

In a ninth aspect, in addition to one or more of the preceding or following aspects, or in the alternative to some aspects, the system can further include a pre-cooler between the first heat exchanger and the working fluid inlet, wherein the pre-cooler can be configured to further cool the working gas to a sub-ambient temperature.

In a tenth aspect, in addition to one or more of the preceding or following aspects, or in the alternative to some aspects, the system can further include a vacuum circuit, wherein the vacuum circuit can be defined within the insulated zone between the return tube and an insulating shaft.

In an eleventh aspect, in addition to one or more of the preceding or following aspects, or in the alternative to some aspects, the compressor can be configured to increase the pressure of the working fluid to at least 3 MPa.

In a twelfth aspect, in addition to one or more of the preceding or following aspects, or in the alternative to some aspects, the system can be configured so that the working fluid experiences a temperature drop of between about 120 Kelvin and 150 Kelvin within the cryoablation probe.

In a thirteenth aspect, a method of operating a closed-loop system for cryosurgery can be included, the method including increasing a pressure of a working fluid in a closed-loop working fluid circuit with a compressor. The method can include, after increasing the pressure of the working fluid, lowering a temperature of the working fluid with a first heat exchanger. The method can include, after lowering the temperature of the working fluid, supplying the working fluid to a cryoablation probe via a working fluid inlet. The cryoablation probe can include a shaft including a supply tube and a return tube surrounding the supply tube, an insulated zone, and an expansion chamber extending distally from the insulated zone. The method can include sending the working fluid to the expansion chamber via the supply tube. The method can include expanding the working fluid in the expansion chamber at a Joule-Thomson orifice, wherein upon expanding at the Joule Thompson orifice, the working fluid cools to a temperature of less than 150 Kelvin. The method can include expelling the expanded working fluid from the cryoablation probe via the return tube and a working fluid outlet, returning the working fluid to the compressor.

In a fourteenth aspect, in addition to one or more of the preceding or following aspects, or in the alternative to some aspects, the working fluid includes a mixed refrigerant.

In a fifteenth aspect, in addition to one or more of the preceding or following aspects, or in the alternative to some aspects, the mixed refrigerant consists of non-flammable and non-toxic components.

In a sixteenth aspect, in addition to one or more of the preceding or following aspects, or in the alternative to some aspects, the working fluid includes a zeotropic mixture.

In a seventeenth aspect, in addition to one or more of the preceding or following aspects, or in the alternative to some aspects, the method can further include storing at least a portion of the working fluid in an accumulator.

In an eighteenth aspect, in addition to one or more of the preceding or following aspects, or in the alternative to some aspects, the accumulator can be configured to maintain a working pressure of the working fluid.

In a nineteenth aspect, in addition to one or more of the preceding or following aspects, or in the alternative to some aspects, the compressor can be configured to increase the pressure of the working fluid to at least 3 MPa.

In a twentieth aspect, in addition to one or more of the preceding or following aspects, or in the alternative to some aspects, the system can be configured so that the working fluid experiences a temperature drop of between about 10 Kelvin and 40 Kelvin when the working fluid expands at the Joule-Thomson orifice.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a closed-loop system for cryosurgery in accordance with various embodiments herein.

FIG. 2 is a perspective view of a cryoablation probe in accordance with various embodiments herein.

FIG. 3 is an enlarged perspective view of the cryoablation probe proximal section of FIG. 2 in accordance with various embodiments herein.

FIG. 4 is an enlarged cross-sectional view of the cryoablation probe distal section shown in FIG. 2 taken along the plane 4-4 in accordance with various embodiments herein.

FIG. 5 is a perspective view of the cryoablation probe distal section shown in FIG. 4 with the outer tube removed from the view in accordance with various embodiments herein.

FIG. 6 is a cross-sectional view of the cryoablation probe shown in FIG. 5 taken along the plane 6-6 in accordance with various embodiments herein.

FIG. 7 is an enlarged perspective view of the cryoablation probe shown in FIG. 2 with the outer tube removed from the view and the insulating shaft and return gas flow lumen sectioned, in accordance with various embodiments herein.

FIG. 8 is a schematic view of components of a closed-loop system for cryosurgery in accordance with various embodiments herein.

FIG. 9 is an exemplary pressure vs. enthalpy plot of a working fluid in the closed-loop working fluid circuit of FIG. 8 in accordance with various embodiments herein.

FIG. 10 is a schematic view of components of a closed-loop system for cryosurgery in accordance with various embodiments herein.

FIG. 11 is a front view of an accumulator in accordance with various embodiments herein.

FIG. 12 is a perspective view of the accumulator of FIG. 11 in accordance with various embodiments herein.

FIG. 13 is a perspective view of certain components of the accumulator of FIG. 11 in accordance with various embodiments herein.

FIG. 14 is a schematic view of a straw component of the accumulator of FIG. 11 in accordance with various embodiments herein.

FIG. 15 is a detailed view of the straw of FIG. 14 about feature 15 in accordance with various embodiments herein.

FIG. 16 is a schematic view of a probe-console connector in accordance with various embodiments herein.

FIG. 17 is a table of exemplary mixed refrigerants in accordance with various embodiments herein.

FIG. 18 is a table of exemplary mixed refrigerants in accordance with various embodiments herein.

While embodiments are susceptible to various modifications and alternative forms, specifics thereof have been shown by way of example and drawings and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Cryoablation systems utilize the expansion of a working fluid to generate a cooling effect. The temperature at the tip of the cryoablation probe can reach cryogenic temperatures (˜140 Kelvin) to form an ice ball and ablate a region in a patient's anatomy. In some embodiments, it is desirable to utilize a cryoablation system having a closed-loop working fluid circuit in which the expanded working fluid is recirculated. For instance, utilizing a closed-loop working fluid circuit can simplify the cryoablation system setup, conserve the working fluid, and allow for cryoablation procedures to be performed in regions where obtaining high-purity, highly pressurized working fluid tanks is difficult or not possible. Moreover, in some embodiments, closed loop cryoablation systems can reduce or eliminate use of large, compressed gas tanks consequently reducing the size and simplifying the setup of the cryoablation system.

A closed-loop system for cryosurgery can include a closed-loop working fluid circuit containing a working fluid. The system can include a compressor configured to increase the pressure of the working fluid, a first heat exchanger configured to lower the temperature of the working fluid, and a cryoablation probe. In various embodiments, after exiting the first heat exchanger, the working fluid is configured to, enter the cryoablation probe via the working fluid inlet, travel to the expansion chamber via the supply tube, expand in the expansion chamber at a Joule-Thomson orifice, travel to the cryoablation probe interface via the return tube, exit the cryoablation probe via the working fluid outlet, enter the compressor downstream from the working fluid outlet, and enter the first heat exchanger downstream from the compressor.

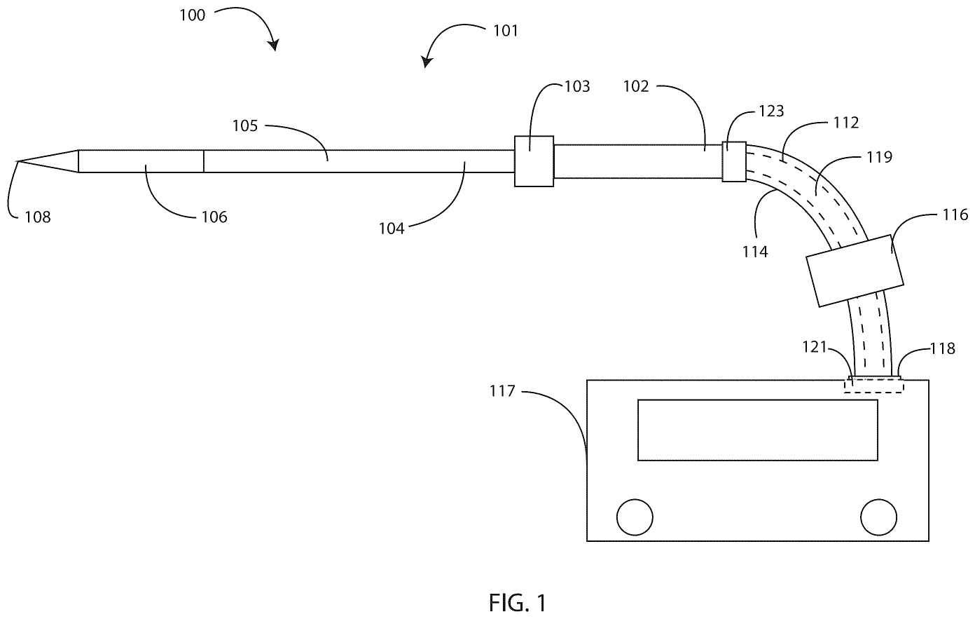

### System Overview (FIG. 1)

Referring now to FIG. 1, a schematic view of a closed-loop system for cryosurgery 100 is shown in accordance with various embodiments herein. In various embodiments, the system can include a cryoablation probe 101 having a handle 102 and a shaft 104. In various embodiments, the shaft 104 is connected to the handle 102 with shaft-handle connector 103. In various embodiments, the shaft 104 and the shaft-handle connector 103 of a closed-loop system for cryosurgery 100 can form a catheter assembly.

In some embodiments, the closed-loop system for cryosurgery 100 includes a console 117. The console may be used to control the system and may be in electrical and fluid communication with the handle and cryoablation assembly. In the example of FIG. 1, the console 117 controls the flow of a working fluid. In various embodiments, the working fluid is contained within a closed-loop working fluid circuit within the cryoablation system. In various embodiments, the cryoablation probe is configured to connect to a cryoablation probe port 121 of the console 117 via a probe-console connector 118.

In the example of FIG. 1, the closed-loop system for cryosurgery 100 can include a line set 119 connecting the console to the handle 102 of the cryoablation probe 101. In various embodiments, the line set 119 is configured to be flexible, enhancing the maneuverability of the cryoablation probe 101. In various embodiments, the closed-loop system for cryosurgery 100 can further include a line set-probe connector 123 configured to form a leak-tight seal between the line set and the cryoablation probe 101.

In the example of FIG. 1, the working fluid is configured to circulate from the console 117 to the cryoablation probe 101 via a working fluid inlet 112 and travel back from the cryoablation probe 101 to the console via a working fluid outlet 114. In the example of FIG. 1, the working fluid outlet 114 shares a common longitudinal axis with the working fluid inlet 112. However, other configurations are possible such as the working fluid inlet 112 being arranged adjacent to the working fluid outlet 114.

In various embodiments, the working fluid circuit runs through both the handle 102 and the shaft 104 of the closed-loop system for cryosurgery 100 and carries the fluid which generates the ice ball. The term “fluid circuit” is used throughout the application, and could be replaced with gas circuit, liquid circuit, fluid chamber, gas chamber, or liquid chamber in various embodiments. The term “fluid” is used throughout and could be replaced with gas or liquid in various embodiments. The working fluid is circulated through the probe to generate an ice ball in the patient's body surrounding an expansion chamber 106. The expansion chamber 106 defines the portion of the shaft 104 that is not insulated to increase thermal conduction and where the ice ball is generated. In various embodiments, the shaft 104 carries high pressure working fluid from the handle 102 to the expansion chamber 106, where it undergoes a Joule-Thomson effect and correspondingly experiences a temperature change. The working fluid exits down the shaft 104, through the handle 102, and continues to circulate through the closed-loop working fluid circuit. In various embodiments, the shaft 104 may include an insulated zone 105. The insulated zone 105 defines the portion of shaft 104 that is insulated to reduce thermal losses and define the shape of the ice ball.

The working fluid can be a cooling fluid or mixture of fluids. In some embodiments, the pressure of the high-pressure stream of the working fluid can be greater than or equal to 2.0 MPa, 2.3 MPa, 2.7 MPa, or 3.0 MPa. In some embodiments, the pressure of the high-pressure stream of the working fluid can be less than or equal to 12.0 MPa, 9.0 MPa, 6.0 MPa, or 3.0 MPa. In some embodiments, the pressure of the high-pressure stream of the working fluid can fall within a range of 2.0 MPa to 12.0 MPa, or 2.3 MPa to 9.0 MPa, or 2.7 MPa to 6.0 MPa, or can be about 3.0 MPa. Pressure measurements are provided as absolute pressure measurements herein.

Accordingly, the temperature of the working fluid at the expansion chamber 106 can be about 140 Kelvin. In some embodiments, the temperature of the working fluid at the expansion chamber 106 can be greater than or equal to 100 Kelvin, 113 Kelvin, 127 Kelvin, or 140 Kelvin. In some embodiments, the temperature of the working fluid at the expansion chamber 106 can be less than or equal to 200 Kelvin, 180 Kelvin, 160 Kelvin, or 140 Kelvin. In some embodiments, the temperature of the working fluid at the expansion chamber 106 can fall within a range of 100 Kelvin to 200 Kelvin, or 113 Kelvin to 180 Kelvin, or 127 Kelvin to 160 Kelvin, or can be about 140 Kelvin.

In various embodiments, the closed-loop system for cryosurgery 100 can further include a heat exchanger 11. The heat exchanger 116 can be positioned in any suitable position within the closed-loop system for cryosurgery 100. In the embodiment of FIG. 1, the heat exchanger 116 is disposed in a hub surrounding a line set 119 connecting the cryoablation probe 101 to the console 12. In various embodiments, the heat exchanger 116 is configured to cool the working fluid prior to the working fluid reaching the expansion chamber 106 of the cryoablation probe 101. The heat exchanger 116 can be any suitable heat exchanger such as a recuperative heat exchanger, an electric cooler, or the like.

In some embodiments, the outer surface of the shaft 104 may be thermally insulated from the inner surface of the shaft. In various embodiments, shaft insulation can be provided by a sealed vacuum chamber or containing a non-circulating fluid or gas within a sealed chamber. In alternative embodiments, a vacuum circuit runs through both the handle 102 and the insulated zone 105 of the shaft 104. Vacuum is actively pulled along the insulated zone 105 of the shaft 104 throughout the cryoablation procedure, providing a protective barrier between the outer surface of the shaft 104 and the patient. In alternative embodiments, shaft insulation can be obtained by circulating fluid, gas, or a heated fluid throughout the shaft or by electrically heating portions of the shaft.

The distal end of the shaft 104 may terminate in a distal operating tip 108. During use, the distal operating tip 108 is deployed in the body of a patient, is surrounded by tissue, and cryogenically ablates the tissue in some instances. The distal operating tip 108 may be advantageously configured to pierce tissue in some instances. For example, the distal operating tip 108 may include a sharp tip, such as a trocar tip. Alternatively, the distal operating tip 108 may not be a sharp tip. In some embodiments, the distal operating tip 108 can be an atraumatic tip designed to cause minimal tissue injury. In some embodiments, the distal operating tip 108 may also contain a working port configured for any of aspiration, delivery of therapeutics, and delivery of other devices including, but not limited to guide wires, imaging catheters, sensing devices, biopsy devices, balloons, and stents.

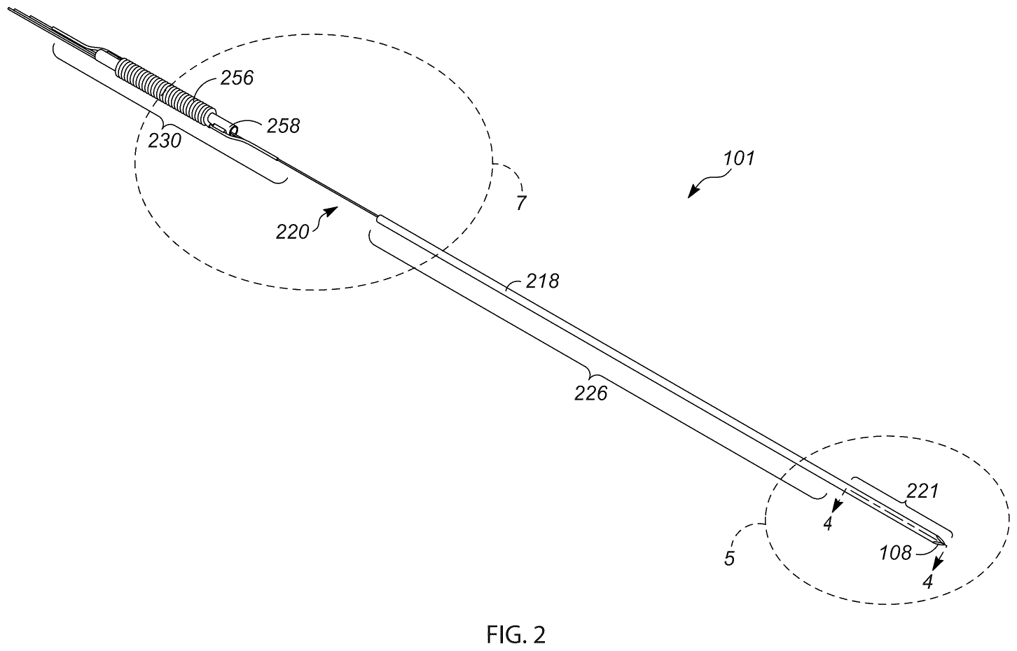

### Cryoablation Probe Configuration (FIGS. 2-7)

The concepts described herein can be applied in the context of the cryoablation probe described in U.S. Pat. No. 11,832,867B2, titled “Cryoneedle” filed Jun. 7, 2019, and incorporated herein by reference in its entirety.

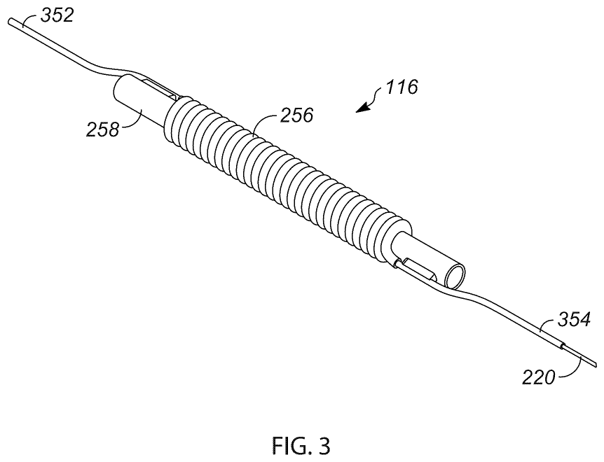

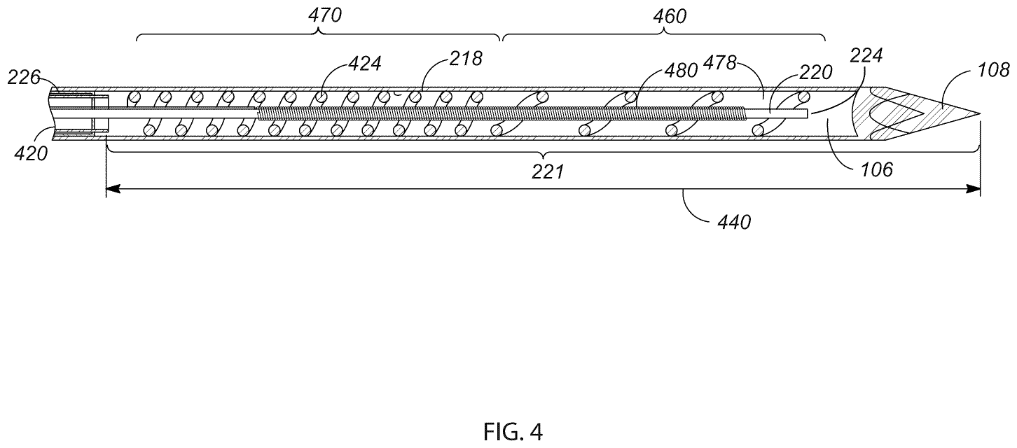

FIGS. 2-7 illustrate an exemplary configuration of a cryoablation probe 101. However, it should be noted that any suitable cryoablation probe can be utilized with the closed-loop system for cryosurgery. A perspective view of a cryoablation probe is shown in FIG. 2 in accordance with various embodiments herein. An enlarged perspective view of the cryoablation probe proximal section of FIG. 2 (identified in detail 5) is shown in FIG. 3 in accordance with various embodiments herein. An enlarged cross-sectional view of the cryoablation probe distal section shown in FIG. 2 (identified in detail 7) taken along the plane 4-4 is shown in FIG. 4 in accordance with various embodiments herein.

As shown in FIGS. 2 and 4, components of the cryoablation probe 101 are located within a return tube 218. The return tube 218 can have a distal operating tip 108 disposed at a distal section 221 of the cryoablation probe 101 for penetrating through tissues of a patient during deployment. The return tube 218 can be of substantially thin cross section for allowing deployment in tissues of a patient. In one example, the return tube 218 has an outer diameter of about 2.1 millimeters (mm). Other dimensions of the return tube 218 are also contemplated. In some embodiments, the outer diameter of the return tube 218 can be greater than or equal to 1.5 mm, 3.7 mm, 5.8 mm, or 8.0 mm. In some embodiments, the outer diameter of the return tube 218 can be less than or equal to 12.0 mm, 10.7 mm, 9.3 mm, or 8.0 mm. In some embodiments, the outer diameter of the return tube 218 can fall within a range of 1.5 mm to 12.0 mm, or 3.7 mm to 10.7 mm, or 5.8 mm to 9.3 mm, or can be about 8.0 mm.

The return tube 218 can also have a middle section 226 positioned between the distal section 221, and a proximal section 230 of the cryoablation probe 101. Additionally, the return tube 218 has a longitudinal axis.

The cryoablation probe 101 includes a supply tube 220 extending substantially along its length for providing a high-pressure gas to the distal operating tip 108. The supply tube 220 can be positioned coaxially/concentrically within the return tube 23. In various embodiments, the supply tube 220 shares a common longitudinal axis with the return tube 218. The supply tube 220 can be configured to supply a working fluid for forming ice balls on an outer surface of the return tube 218 over the distal section 221. In some cases, the supply tube 220 can be a capillary tube. Referring to FIG. 3, the cryoablation probe includes a heat exchanger 116 positioned in the proximal section 230 of the cryoablation probe 101 (shown in FIG. 2). The heat exchanger 116 can be operably connected to the working fluid circuit at a first end 352 and the supply tube 220 at a second end 354. The heat exchanger 116 can precool the working fluid from the working fluid circuit prior to being delivered to the middle and/or distal sections.

With continued reference to FIG. 3, the heat exchanger 116 can take the form of a heat exchanger having a hollow helical tube 256 wound around a central core 258. The helical tube 256 of the heat exchanger 116 provides increased heat exchange surface area per unit length of the helical tube 256 in comparison to heat exchangers that are not coiled. The helical tube 256 can be made of brass. Other metals such as stainless steel are also contemplated. In the illustrated embodiment shown in FIG. 2, the heat exchanger 116 is distanced from the distal operating tip 108. For instance, the heat exchanger 116 can be positioned at a handle of the cryoablation probe (not shown in this view).

The heat exchanger 116 can be positioned in any suitable position within the closed-loop system for cryosurgery 100. In some embodiments, the heat exchanger 116 is positioned immediately proximal to the shaft 104 (e.g., abutting the proximal end of the shaft). In some embodiments, the heat exchanger 116 is positioned further proximal to the shaft 104 and additional tubing is added to connect the heat exchanger 116 to the shaft. In some embodiments, the heat exchanger 116 is disposed in a hub or docking station that is configured to connect to multiple cryoablation probes.

Referring to FIG. 4, the distal section 221 can have a first portion 460 and a second portion 470. The cryoablation probe 101 can include an expansion chamber 106 within the first portion 460 of the distal section 221 such that the supply tube 220 terminates in the expansion chamber 106. In some cases, the supply tube 220 (e.g., capillary tube) can terminate in a Joule-Thomson orifice 224. The Joule-Thomson orifice 224 can be positioned near the expansion chamber 106. A high-pressure working fluid supplied via the supply tube 220 exits supply tube 220 via the Joule-Thomson orifice 224 and expands in the expansion chamber 106. As the working fluid expands in the expansion chamber 106, it cools rapidly and forms ice balls of different shapes and/or sizes over the outer surface of the return tube 218. The expansion of the working fluid can be such that when expanded, the working fluid in the expansion chamber 106 has a reduced temperature than that of the incoming working fluid.

With continued reference to FIG. 4, the cryoablation probe 101 can include a return gas flow lumen 478 defined between the supply tube 220 and the return tube 218 of the cryoablation probe 101. In certain embodiments, the return gas flow lumen 478 is helical in shape.

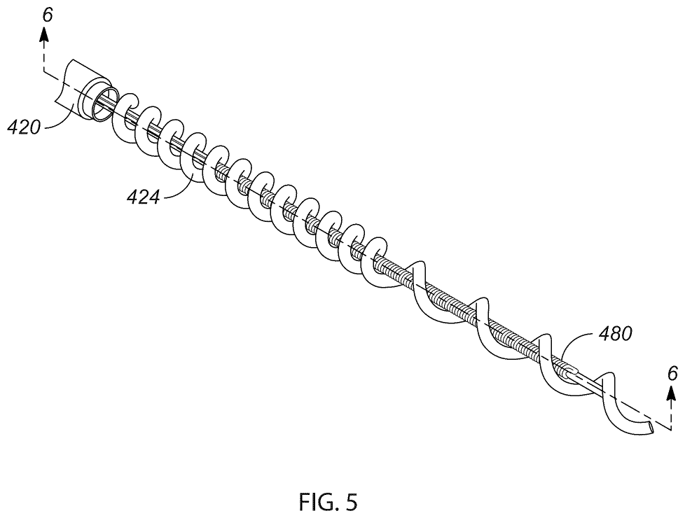

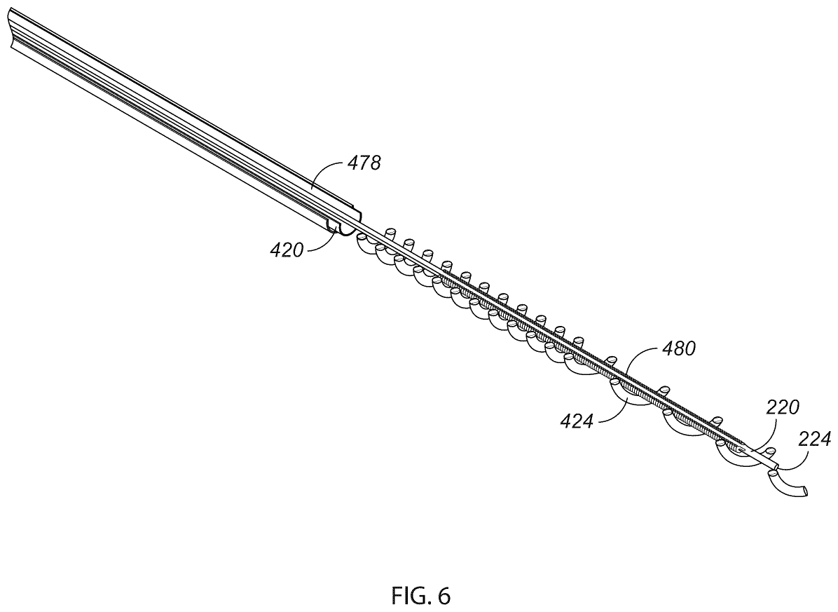

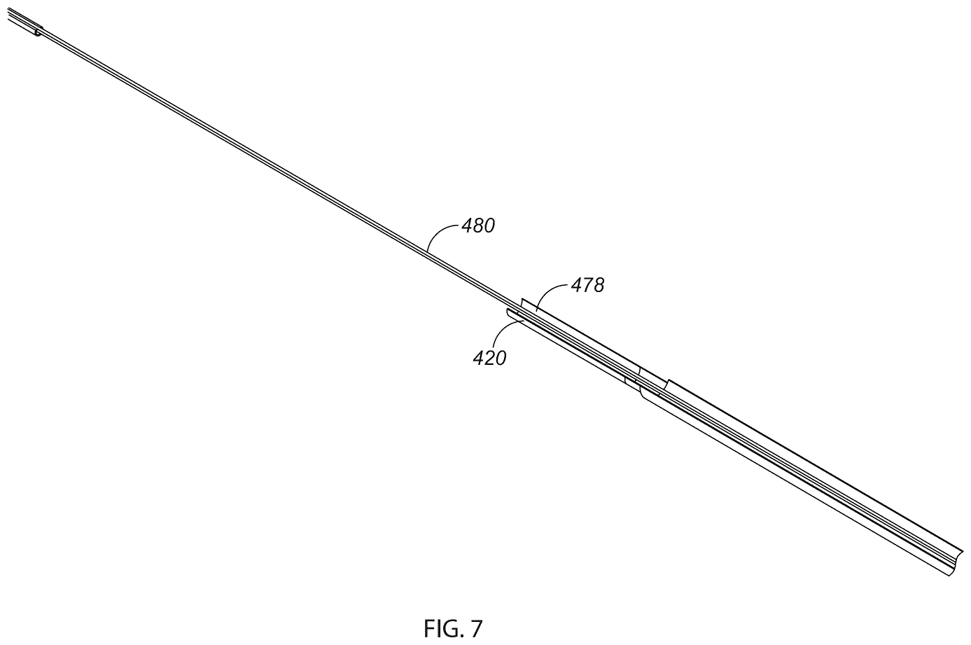

A perspective view of the cryoablation probe shown in FIG. 2 about detail 5 with the outer tube removed from the view is shown in FIG. 5 in accordance with various embodiments herein. A cross-sectional view of the cryoablation probe shown in FIG. 5 taken along the plane 6-6 is shown in FIG. 6 in accordance with various embodiments herein. An enlarged perspective view of the cryoablation probe distal section shown in FIG. 2 about detail 7 with the outer tube removed from the view is shown FIG. 7 in accordance with various embodiments herein.

Referring to FIGS. 4-7, in some cases, a heater 480 can optionally be provided within the return tube 218 near the distal section 221 to facilitate disengagement of cryoablation probe 101 after ablating a tissue, for thawing a tissue at or near the distal section 221 of the cryoablation probe 101, for cauterizing tissue, or for other purposes. As illustrated in this exemplary embodiment, an electrical heater 480 is provided coaxially with the supply tube 220 and the return tube 218 to facilitate heating the distal section 221 of the cryoablation probe 101. Alternatively, the electrical heater 480 can be positioned elsewhere in cryoablation probe 101 to heat the distal section 221 of the cryoablation probe 101.

As mentioned previously, (referring to FIG. 4) the temperature of the working fluid can be reduced in the first portion 460 of the distal section 221 (due to Joule-Thomson effect) compared to in the second portion 470, and heat transfer between the working fluid and the return tube 218 can be higher in magnitude over the first portion 460 than in the second portion 470. This can result in an ice ball having an asymmetric shape (e.g., pear-shaped with the ice ball generally tapering toward the second portion 470). To prevent asymmetric ice ball formation, as seen in FIGS. 4-6, in one example, the cryoablation probe 101 includes a heat exchange helix 424, comprising coils configured for enhancing heat exchange between the working fluid and the return tube 218. The heat exchange helix 424 contacts the inner wall of the return tube 218 and is positioned coaxially with the return tube 218. As seen in FIG. 4, the coils of the heat exchange helix 424 are in contact with the inner wall of the return tube 218 and do not contact the heater 480 or the supply tube 220 (e.g., capillary tube). These coils effectively act as fins on the inner wall of the return tube 218 and improve heat transfer to the return tube 218 to form ice balls having symmetric shapes wherein the symmetry is about the longitudinal axis of the return tube 218.

Referring to FIGS. 4-7, the cryoablation probe 101 comprises an insulating shaft 420. The insulating shaft 420 abuts against the return tube 218 forming the insulated zone 105 of the cryoablation probe 101. The insulating shaft 420 is configured to confine the formation of the ice ball to the expansion chamber 106 of the cryoablation probe 101. In the illustrated embodiment shown in FIG. 4, the entire length 440 of the distal section 221 is exposed. Over this exposed region length 440, the expanded working fluid contacts the return tube 218 of the cryoablation probe 101 and forms an ice ball. The insulating shaft 420 is positioned over the entire length of the middle section 226 of the cryoablation probe 101 where the working fluid is prevented from contacting the return tube 218, thereby preventing cooling of tissue surrounding the middle section 226 and/or ice ball formation thereon. In alternate embodiments, a vacuum can be actively pulled along the insulated zone 105 of the shaft 104.

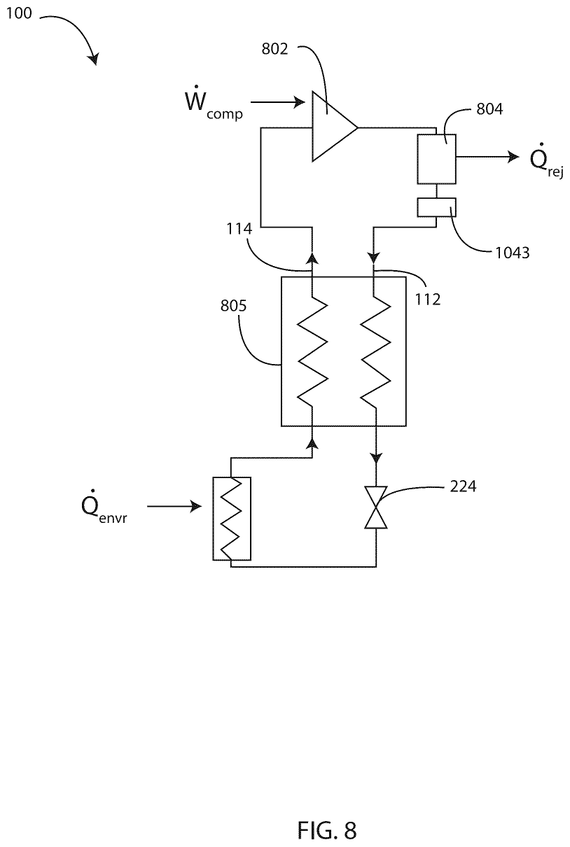

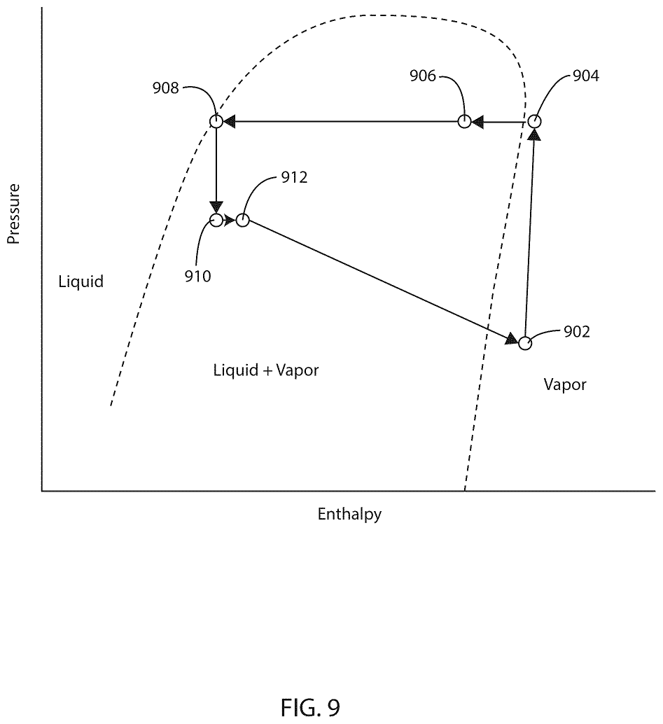

### Closed-Loop Working Fluid Circuit (FIGS. 8-9)

FIGS. 8-9 illustrate an example of a closed-loop system for cryosurgery having a closed-loop working fluid circuit. A schematic view of components of a closed-loop system for cryosurgery is shown in FIG. 8 in accordance with embodiments herein. An exemplary pressure vs. enthalpy plot of a working fluid in the closed-loop working fluid circuit of FIG. 8 is shown in FIG. 9 with various embodiments herein.

A closed-loop fluid circuit as defined herein is a system where a working fluid circulates continuously in a sealed loop. The closed-loop fluid circuit is configured to recirculate the working fluid, minimizing losses and contamination. In various embodiments, the closed-loop fluid circuit does not vent the working fluid to the external environment.

In various embodiments, the working fluid is configured to continuously circulate about the closed-loop working fluid circuit. For this example, a particular segment of the working fluid in the closed-loop working fluid circuit will be analyzed beginning at the compressor (corresponding to the compressor 802 in FIG. 8 and point 902 in FIG. 9).

In various embodiments, the closed-loop system for cryosurgery 100 can include a compressor 802 configured to increase the pressure of the working fluid. Referring now to FIG. 9, the working fluid enters the compressor 802 at point 902 having a first pressure. After being compressed by the compressor, the working fluid exits the compressor 802 at point 904 having a second pressure that is greater than the first pressure.

In some embodiments, the pressure of the working fluid at point 902 can be greater than or equal to 0.05 MPa, 0.10 MPa, 0.15 MPa, or 0.20 MPa. In some embodiments, the pressure of the working fluid at point 902 can be less than or equal to 0.50 MPa, 0.40 MPa, 0.30 MPa, or 0.20 MPa. In some embodiments, the pressure of the working fluid at point 902 can fall within a range of 0.05 MPa to 0.50 MPa, or 0.10 MPa to 0.40 MPa, or 0.15 MPa to 0.30 MPa, or can be about 0.20 MPa.

In some embodiments, the temperature of the working fluid at point 902 can be greater than or equal to 263 Kelvin, 266 Kelvin, 270 Kelvin, or 273 Kelvin. In some embodiments, the temperature of the working fluid at point 902 can be less than or equal to 283 Kelvin, 280 Kelvin, 276 Kelvin, or 273 Kelvin. In some embodiments, the temperature of the working fluid at point 902 can fall within a range of 263 Kelvin to 283 Kelvin, or 266 Kelvin to 280 Kelvin, or 270 Kelvin to 276 Kelvin, or can be about 273 Kelvin.

In some embodiments, the pressure of the working fluid at point 904 can be greater than or equal to 2.0 MPa, 2.4 MPa, 2.7 MPa, or 3.1 MPa. In some embodiments, the pressure of the working fluid at point 904 can be less than or equal to 5.0 MPa, 4.4 MPa, 3.7 MPa, or 3.1 MPa. In some embodiments, the pressure of the working fluid at point 904 can fall within a range of 2.0 MPa to 5.0 MPa, or 2.4 MPa to 4.4 MPa, or 2.7 MPa to 3.7 MPa, or can be about 3.1 MPa.

In some embodiments, the temperature of the working fluid at point 904 can be greater than or equal to 290 Kelvin, 300 Kelvin, 310 Kelvin, or 320 Kelvin. In some embodiments, the temperature of the working fluid at point 904 can be less than or equal to 350 Kelvin, 340 Kelvin, 330 Kelvin, or 320 Kelvin. In some embodiments, the temperature of the working fluid at point 904 can fall within a range of 290 Kelvin to 350 Kelvin, or 300 Kelvin to 340 Kelvin, or 310 Kelvin to 330 Kelvin, or can be about 320 Kelvin.

In some embodiments, the pressure increase to the working fluid caused by the compressor 802 can be greater than or equal to 0.5 MPa, 1.4 MPa, 2.2 MPa, or 3.1 MPa. In some embodiments, the pressure increase to the working fluid caused by the compressor 802 can be less than or equal to 6.0 MPa, 5.0 MPa, 4.1 MPa, or 3.1 MPa. In some embodiments, the pressure increase to the working fluid caused by the compressor 802 can fall within a range of 0.5 MPa to 6.0 MPa, or 1.4 MPa to 5.0 MPa, or 2.2 MPa to 4.1 MPa, or can be about 3.1 MPa.

In some embodiments, after exiting the compressor 802, the temperature of the working fluid has increased to above ambient temperature. In such embodiments, it can be desirable to cool the heated working fluid back down to approximately ambient temperature or lower. In various embodiments, the closed-loop system for cryosurgery 100 can include a first heat exchanger 804 downstream from the compressor. In various embodiments, the first heat exchanger 804 is configured to lower the temperature of the working fluid.

Referring now to FIG. 9, the working fluid enters the first heat exchanger 804 at point 904 having a first temperature. After being cooled by the first heat exchanger 804, the working fluid exits the first heat exchanger 804 at point 906 having a second temperature that is lower than the first temperature. As can further be seen by FIG. 9, while being cooled by the first heat exchanger 804, the working fluid transitions from its vapor phase to a mixed liquid/vapor phase.

In some embodiments, the closed-loop system for cryosurgery 100 can include an additional pre-cooler 1043 between the first heat exchanger 804 and the cryoablation probe 101. After being cooled by the first heat exchanger 804 and the pre-cooler 1043, the working fluid exits the pre-cooler 1043 at point 906 having a second temperature that is lower than the first temperature. In some embodiments, the additional pre-cooler is configured to cool the refrigerant mixture to a sub-ambient temperature before the working fluid enters the cryoablation probe 101.

In some embodiments, the pressure of the working fluid at point 906 can be greater than or equal to 2.0 MPa, 2.4 MPa, 2.7 MPa, or 3.1 MPa. In some embodiments, the pressure of the working fluid at point 906 can be less than or equal to 5.0 MPa, 4.4 MPa, 3.7 MPa, or 3.1 MPa. In some embodiments, the pressure of the working fluid at point 906 can fall within a range of 2.0 MPa to 5.0 MPa, or 2.4 MPa to 4.4 MPa, or 2.7 MPa to 3.7 MPa, or can be about 3.1 MPa.

In some embodiments, the temperature of the working fluid at point 906 can be greater than or equal to 250 Kelvin, 263 Kelvin, 277 Kelvin, or 290 Kelvin. In some embodiments, the temperature of the working fluid at point 906 can be less than or equal to 310 Kelvin, 303 Kelvin, 297 Kelvin, or 290 Kelvin. In some embodiments, the temperature of the working fluid at point 906 can fall within a range of 250 Kelvin to 310 Kelvin, or 263 Kelvin to 303 Kelvin, or 277 Kelvin to 297 Kelvin, or can be about 290 Kelvin.

In some embodiments, the temperature decrease to the working fluid caused by the first heat exchanger 804 can be greater than or equal to 10 Kelvin, 17 Kelvin, 23 Kelvin, or 30 Kelvin. In some embodiments, the temperature decrease to the working fluid caused by the first heat exchanger 804 can be less than or equal to 50 Kelvin, 43 Kelvin, 37 Kelvin, or 30 Kelvin. In some embodiments, the temperature decrease to the working fluid caused by the first heat exchanger 804 can fall within a range of 10 Kelvin to 50 Kelvin, or 17 Kelvin to 43 Kelvin, or 23 Kelvin to 37 Kelvin, or can be about 30 Kelvin.

In the example of FIGS. 8-9, the first heat exchanger 804 itself does not have sufficient cooling capacity to drive the working fluid to cryogenic temperatures (˜140 Kelvin). In various embodiments, after exiting the first heat exchanger, the working fluid is configured to enter the cryoablation probe 101 via the working fluid inlet 112 and travel to the expansion chamber 106 via the supply tube 220. In various embodiments, the incoming stream of working fluid is cooled as it travels up the supply tube 220 to the expansion chamber 106. As described in the context of FIGS. 2-3 the heat exchanger 116 of the cryoablation probe 101 is configured to cool the incoming stream of working fluid. Moreover, after expanding at the Joule-Thomson orifice 224, the working fluid exiting the cryoablation probe 101 via the return tube 218 is configured to cool the incoming stream of working fluid, forming a recuperative heat exchanger 805.

Referring now to FIG. 9, the working fluid enters the cryoablation probe 101 at point 906 having a first temperature. After being cooled by the various heat exchange mechanisms of the cryoablation probe 101, the working fluid approaches the Joule-Thomson orifice 224 within the expansion chamber 106 of the cryoablation probe 101 at point 908 having a second temperature that is lower than the first temperature. Moreover, as the working fluid is cooled by the cryoablation probe 101, the amount of liquid in the mixed liquid/vapor phase working fluid increases.

In some embodiments, the pressure of the working fluid at point 908 can be greater than or equal to 2.0 MPa, 2.4 MPa, 2.7 MPa, or 3.1 MPa. In some embodiments, the pressure of the working fluid at point 908 can be less than or equal to 5.0 MPa, 4.4 MPa, 3.7 MPa, or 3.1 MPa. In some embodiments, the pressure of the working fluid at point 908 can fall within a range of 2.0 MPa to 5.0 MPa, or 2.4 MPa to 4.4 MPa, or 2.7 MPa to 3.7 MPa, or can be about 3.1 MPa.

In some embodiments, the temperature of the working fluid at point 908 can be greater than or equal to 120 Kelvin, 132 Kelvin, 143 Kelvin, or 155 Kelvin. In some embodiments, the temperature of the working fluid at point 908 can be less than or equal to 200 Kelvin, 185 Kelvin, 170 Kelvin, or 155 Kelvin. In some embodiments, the temperature of the working fluid at point 908 can fall within a range of 120 Kelvin to 200 Kelvin, or 132 Kelvin to 185 Kelvin, or 143 Kelvin to 170 Kelvin, or can be about 155 Kelvin.

In some embodiments, the temperature decrease to the working fluid by the cryoablation probe 101 can be greater than or equal to 100 Kelvin, 112 Kelvin, 123 Kelvin, or 135 Kelvin. In some embodiments, the temperature decrease to the working fluid by the cryoablation probe 101 can be less than or equal to 175 Kelvin, 162 Kelvin, 148 Kelvin, or 135 Kelvin. In some embodiments, the temperature decrease to the working fluid by the cryoablation probe 101 can fall within a range of 100 Kelvin to 175 Kelvin, or 112 Kelvin to 162 Kelvin, or 123 Kelvin to 148 Kelvin, or can be about 135 Kelvin.

After travelling to the expansion chamber 106 via the supply tube 220, the working fluid is configured to expand in the expansion chamber at the Joule-Thomson orifice 224. Referring now to FIG. 9, the expansion of the working fluid at the Joule-Thomson orifice 224 begins at point 908 and ends at point 910. Upon expanding at the Joule-Thomson orifice 224, the working fluid cools to a temperature of less than 150 Kelvin and experiences isenthalpic expansion (no change to enthalpy) as it exits the supply tube 220 at the Joule-Thomson orifice 224. As described in the context of FIGS. 2-7, as the working fluid expands at the Joule-Thomson orifice 224, ice ball formation occurs.

In some embodiments, the pressure of the working fluid at point 910 can be greater than or equal to 0.1 MPa, 0.3 MPa, 0.6 MPa, or 0.8 MPa. In some embodiments, the pressure of the working fluid at point 910 can be less than or equal to 1.5 MPa, 1.3 MPa, 1.0 MPa, or 0.8 MPa. In some embodiments, the pressure of the working fluid at point 910 can fall within a range of 0.1 MPa to 1.5 MPa, or 0.3 MPa to 1.3 MPa, or 0.6 MPa to 1.0 MPa, or can be about 0.8 MPa.

In some embodiments, the temperature of the working fluid at point 910 can be greater than or equal to 120 Kelvin, 123 Kelvin, 127 Kelvin, or 130 Kelvin. In some embodiments, the temperature of the working fluid at point 910 can be less than or equal to 150 Kelvin, 143 Kelvin, 137 Kelvin, or 130 Kelvin. In some embodiments, the temperature of the working fluid at point 910 can fall within a range of 120 Kelvin to 150 Kelvin, or 123 Kelvin to 143 Kelvin, or 127 Kelvin to 137 Kelvin, or can be about 130 Kelvin.

Upon expanding at the Joule-Thomson orifice 224 and forming an ice ball, the working fluid absorbs heat from the surrounding environment (e.g., the tissue of a patient). Referring now to FIG. 9, the absorption of heat from the surrounding environment begins at point 910 and ends at point 912. Upon absorbing heat from the surrounding environment, the temperature of the working fluid increases from point 910 to point 912.

In some embodiments, the pressure of the working fluid at point 912 can be greater than or equal to 0.1 MPa, 0.3 MPa, 0.6 MPa, or 0.8 MPa. In some embodiments, the pressure of the working fluid at point 912 can be less than or equal to 1.5 MPa, 1.3 MPa, 1.0 MPa, or 0.8 MPa. In some embodiments, the pressure of the working fluid at point 912 can fall within a range of 0.1 MPa to 1.5 MPa, or 0.3 MPa to 1.3 MPa, or 0.6 MPa to 1.0 MPa, or can be about 0.8 MPa.

In some embodiments, the temperature of the working fluid at point 912 can be greater than or equal to 120 Kelvin, 127 Kelvin, 133 Kelvin, or 140 Kelvin. In some embodiments, the temperature of the working fluid at point 912 can be less than or equal to 170 Kelvin, 160 Kelvin, 150 Kelvin, or 140 Kelvin. In some embodiments, the temperature of the working fluid at point 912 can fall within a range of 120 Kelvin to 170 Kelvin, or 127 Kelvin to 160 Kelvin, or 133 Kelvin to 150 Kelvin, or can be about 140 Kelvin.

After absorbing heat from the surrounding environment, the working fluid is configured to exit the cryoablation probe 101 via the return tube 218 and the working fluid outlet 114. As previously described, the returning stream working fluid is configured to cool the incoming stream of working fluid via recuperative heat transfer. Accordingly, the returning stream of working fluid absorbs heat from the incoming stream of working fluid. After exiting the cryoablation probe 101 via the return tube 218 and the working fluid outlet 114, the working fluid is configured to return to the compressor 802.

Referring now to FIG. 9, the working fluid begins its return to the compressor 802 via the return tube 218 of the cryoablation probe 101 at point 912 having a first pressure and first temperature. Upon exiting the cryoablation probe 101 via the working fluid outlet 114, the working fluid returns to approximately its initial pressure and temperature values corresponding to point 902. The working fluid can then return to the compressor 802 and recirculate about the closed-loop working fluid circuit, repeating the thermodynamic cycle.

In some embodiments, it may be desirable to increase the pressure of the working fluid delivered to the Joule-Thomson orifice 224 of the cryoablation probe 101. In such configurations, the compressor 802 may be configured to pressurize the working fluid to a pressure of greater than or equal to 3 MPa, 5 MPa, 7 MPa, 9 MPa, 10 MPa, 12 MPa, or greater.

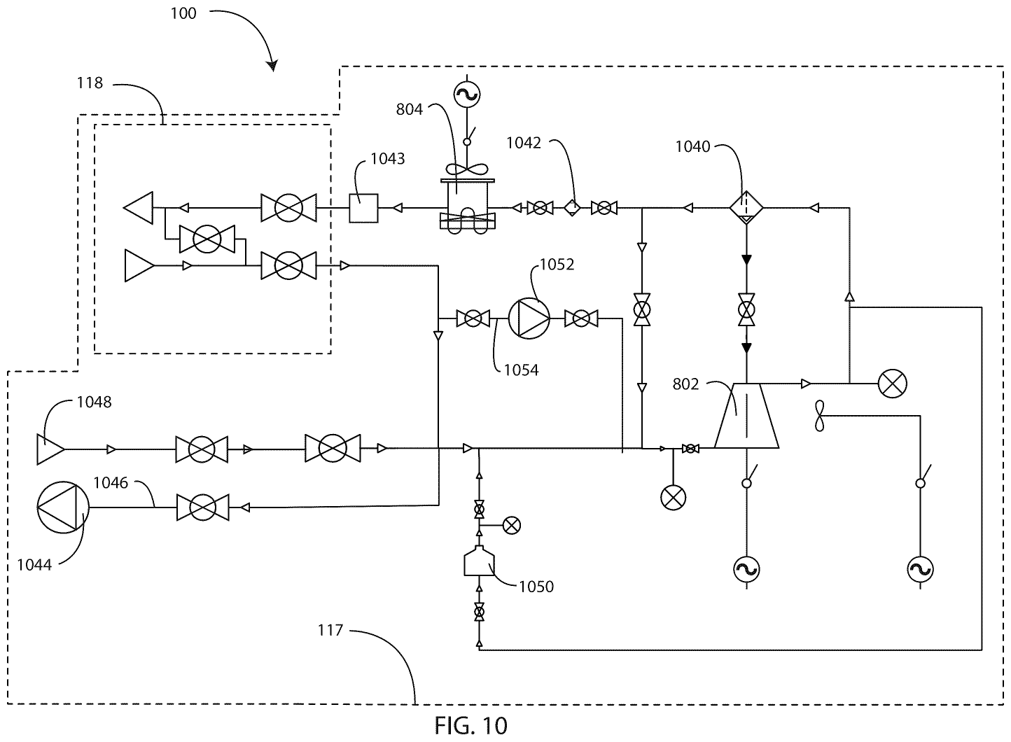

### Detailed Schematic of the Closed-Loop Working Fluid Circuit (FIG. 10)

Referring now to FIG. 10, a schematic view of components of a closed-loop system for cryosurgery is shown in accordance with embodiments herein. The closed-loop system for cryosurgery of FIG. 10 is included to illustrate an exemplary implementation of the closed-loop system for cryosurgery and thermodynamic cycle shown in and described by FIGS. 8-9.

In the example of FIG. 10, various components of the closed-loop system for cryosurgery 100 can be housed within the console 117 of the closed-loop system for cryosurgery 100. Alternatively, any of the components of the closed-loop system for cryosurgery 100 can be external to the console 117.

In various embodiments, the closed-loop system for cryosurgery 100 can include a compressor 802 configured to increase the pressure of the working fluid. The compressor 802 can be any suitable type of compressor such as a positive displacement compressor, dynamic compressor or the like.

In some embodiments, the compressor 802 may contain oil for purposes of cooling, lubrication, or the like. In such embodiments, the closed-loop system for cryosurgery 100 can further include an oil separator 1040 downstream from the compressor 802. In various embodiments, the oil separator 1040 is configured to remove the oil from the compressed working fluid prior to the working fluid entering the cryoablation probe 101. The oil separator can be any suitable type of oil separator such as a gravity oil separator, centrifugal separator, or the like.

In some embodiments, the closed-loop system for cryosurgery 100 can include a filter-dryer 1042 downstream from the compressor. In various embodiments, the filter-dryer 1042 is configured to separate any solid particles from the working fluid prior to the working fluid entering the cryoablation probe 101. The filter-dryer 1042 can be a Nutsche filter-dryer, or the like.

In various embodiments, the closed-loop system for cryosurgery 100 can include a first heat exchanger 804 downstream from the compressor. In various embodiments, the first heat exchanger 804 is configured to lower the temperature of the working fluid. The first heat exchanger 804 can be an axial fan aftercooler, or the like. As described in the context of FIGS. 8-9, the first heat exchanger 804 is configured to cool the working fluid to approximately ambient temperature.

In some embodiments, the closed-loop system for cryosurgery 100 can include an additional pre-cooler 1043 between the first heat exchanger 804 and the cryoablation probe 101. In various embodiments, the pre-cooler 1043 is configured to cool the refrigerant mixture to a sub-ambient temperature before the working fluid enters the cryoablation probe 101. The inclusion of a pre-cooler 1043 in the closed-loop system for cryosurgery 100 can reduce the amount of heat required to be removed from the working fluid in the cryoablation probe 101 by recuperative heat transfer within the cryoablation probe 101. In alternate configurations, the first heat exchanger 804 has the cooling power to cool the working fluid to sub-ambient temperatures negating the need for an additional pre-cooler.

In the example of FIG. 10, the pre-cooler 1043 is positioned downstream from the first heat exchanger 804. In some configurations, the pre-cooler 1043 can be integrated into the first heat exchanger 804. For instance, the first heat exchanger 804 can include multiple cooling loops with the pre-cooler 1043 taking the form of one more of the subsequent cooling loops. In some configurations, the first heat exchanger 804 can accomplish a transition from above ambient temperature to below ambient temperature without an additional pre-cooler 1043.

In various embodiments, the closed-loop system for cryosurgery 100 can include a probe-console connector 118 configured to connect a cryoablation probe 101 to the console. As shown in FIGS. 8-9, the working fluid is configured to enter the cryoablation probe 101 via the working fluid inlet 112 and travel to the expansion chamber 106 via the supply tube 220. After travelling to the expansion chamber 106 via the supply tube 220, the system is configured so that working fluid expands in the expansion chamber at the Joule-Thomson orifice 224, absorbs heat from the surrounding environment (e.g., the tissue of a patient), and causes an ice ball to form. After absorbing heat from the surrounding environment, the working fluid is configured to exit the cryoablation probe 101 via the return tube 218 and the working fluid outlet 114.

In various embodiments, the closed-loop system for cryosurgery 100 can include a recharge line 1048. In various embodiments, the recharge line can be connected to a working fluid source. Additional working fluid can be added to the working fluid circuit via the recharge line 1048.

In various embodiments, the closed-loop system for cryosurgery 100 can include a first vacuum pump 1044 in fluid communication with a first vacuum line 1046. In various embodiments, the first vacuum pump 1044 is configured to evacuate the air from the cryoablation system. The first vacuum pump 1044 can be configured to evacuate the air from the cryoablation system for any number of scenarios such as prior to adding to the working fluid circuit via the recharge line 1048 and/or prior to performing a cryoablation procedure.

In various embodiments, the closed-loop system for cryosurgery 100 can include a second vacuum pump 1052 in fluid communication with a second vacuum line 1054. In scenarios, such as after a cryoablation procedure is complete, it is desirable for the working fluid to be recaptured into the console 117. In such scenarios, a system operator can turn off the compressor 802 and turn on the second vacuum pump 1052 resulting in the second vacuum pump 1052 recovering the working fluid out from the cryoablation probe and into the compressor 802.

In various embodiments, the closed-loop system for cryosurgery 100 can include an accumulator 1050, which will be shown and described in greater detail herein.

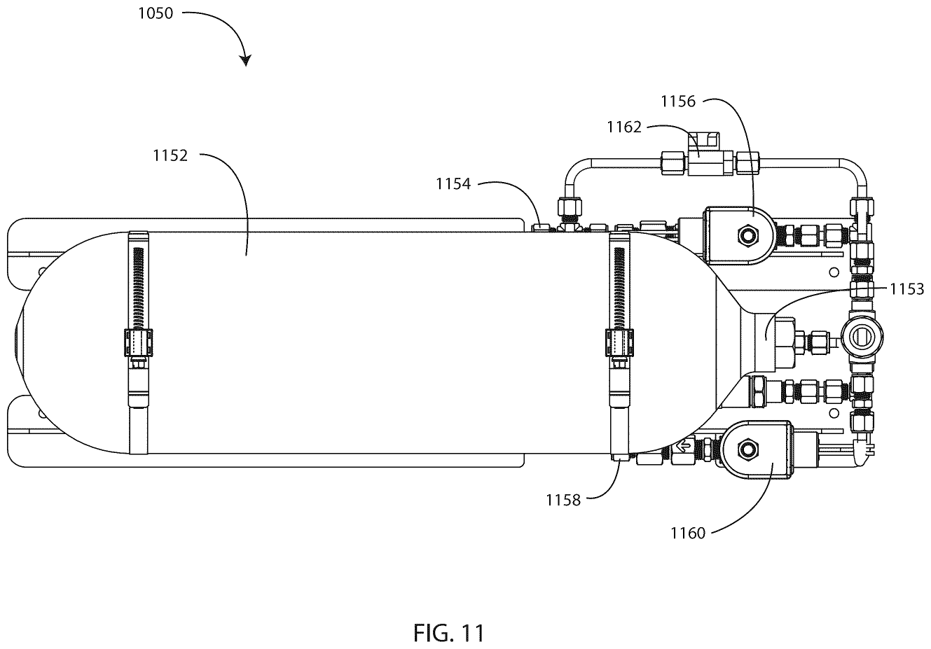

### Accumulator (FIGS. 11-15)

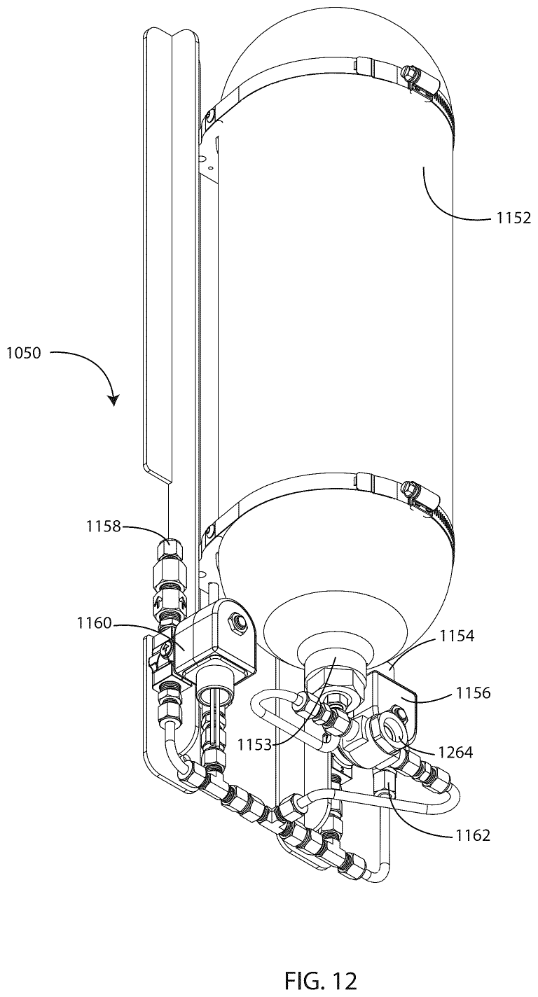

Referring now to FIGS. 11-12, exemplary views of an accumulator are shown in accordance with embodiments herein. FIG. 11 depicts a front view of an accumulator as shown in accordance with embodiments herein. FIG. 12 depicts a perspective view of an accumulator as shown in accordance with embodiments herein. An accumulator as defined herein is a device configured to store pressurized fluid. The accumulator 1050 is configured to serve as a reservoir that can absorb excess fluid, release it when needed, and help maintain pressure and flow within the closed-loop system for cryosurgery 100.

In various embodiments, the accumulator 1050 can include a storage tank 1152 having a tank opening 1153, an inlet 1154 in fluid communication with an inlet valve 1156, and an outlet 1158 in fluid communication with an outlet valve 1160. In various embodiments, the inlet valve 1156 and the outlet valve 1160 can each be manually or remotely controlled orifices. The orifices are configured to limit the flow rate of the refrigerant into and out of the accumulator 1050.

In various embodiments, the accumulator 1050 is configured to control the pressure of the working fluid in the closed-loop system for cryosurgery 100. In particular, the accumulator 1050 is configured to control the pressure of the incoming stream working fluid (e.g., the compressed working fluid travelling to the expansion chamber 106 of the cryoablation probe 101 via the supply tube 220). In some embodiments, the accumulator 1050 is configured to maintain the incoming stream working fluid at a substantially constant pressure.

At the beginning of a cryoablation cycle, the compressor 802 and the working fluid are at approximately room temperature, resulting in a relatively low pressure increase of the working fluid. As described in the context of FIGS. 8-9, the compressor 802 can raise both the temperature and the pressure of the working fluid. Accordingly, as the working fluid and the compressor 802 warm up during the cryoablation cycle, the compressor 802 becomes more effective in pressurizing the working fluid, resulting in a higher pressure for the incoming stream working fluid.

In various embodiments, it is desirable to maintain a stable fluid pressure in the incoming stream of working fluid. In various embodiments, the accumulator is configured to counteract the increase in pressure of the incoming stream of working fluid during a cryoablation cycle. For instance, the accumulator 1050 is configured to temporarily store a portion of the working fluid in the storage tank 1152. As the pressure of the incoming stream of working fluid increases during a cryoablation cycle, the accumulator 1050 can actuate the inlet valve 1156 causing a portion of the working fluid to enter the accumulator 1050 via the inlet 1154 and be temporarily stored in the storage tank 1152 via the tank opening 1153. Storing a portion of working fluid in the accumulator reduces the amount of working fluid circulating through the closed-loop working fluid circuit and thus lowers the pressure of the incoming stream of working fluid.

In various embodiments, the accumulator 1050 can include one or more check valves configured to prevent backflow of the working fluid. In various embodiments, the accumulator 1050 can include one or more pressure gauges and/or transducers configured to provide information on the amount of working fluid in the storage tank 1152. In various embodiments, the closed-loop system for cryosurgery 100 is configured to monitor the pressure of the incoming stream of working fluid and the amount of working fluid in the storage tank 1152. Based on this information, the closed-loop system for cryosurgery 100 can be further configured to actuate the inlet valve 1156 and outlet valve 1160 of the accumulator to adjust the amount of working fluid in the storage tank 1152 (and thus the amount of working fluid circulating through the closed-loop working fluid circuit) and maintain a constant pressure in the incoming stream of working fluid.

In various embodiments, the accumulator 1050 is further configured to adjust the working fluid pressure to accommodate different types of cryoablation probes. For instance, cryoablation probes with larger supply tubes use more working gas to maintain the same pressure drop at the Joule-Thomson orifice. Accordingly, the accumulator 1050 can be configured to store more working fluid in the storage tank 1152 for a cryoablation probe with a smaller supply tube and less working fluid in the storage tank for a cryoablation probe with a larger supply tube.

In various embodiments, the accumulator 1050 is further configured to adjust the working fluid pressure to accommodate different types of working fluids. For instance, some working fluids may require a smaller pressure drop to generate an ice ball than other working fluids. Accordingly, the accumulator 1050 can be configured to store more working fluid in the storage tank 1152 for a working fluid requiring a smaller pressure drop and less working fluid in the storage tank 1152 for a working fluid requiring a larger pressure drop.

In various embodiments, the accumulator 1050 can further include an additional valve 1162. In the example of FIG. 11, the valve 1162 is manually operated. Alternatively, the valve 1162 can be automatically controlled. In various embodiments, the valve 1162 is normally closed but can be opened for servicing of the accumulator 1050. In various embodiments, the valve 1162 is in fluid communication with a reservoir of refrigerant and can be opened for the charging and/or discharging of refrigerant from the storage tank 1152 of the accumulator 1050.

In various embodiments, the accumulator 1050 can further include a condensation monitor 1264. The condensation monitor can be any suitable type of condensation monitor such as an oil sight glass level monitor, or the like. In various embodiments, certain components of the closed-loop system for cryosurgery 100 may contain oil and the condensation monitor 1264 is configured to detect the presence of oil within the accumulator 1050.

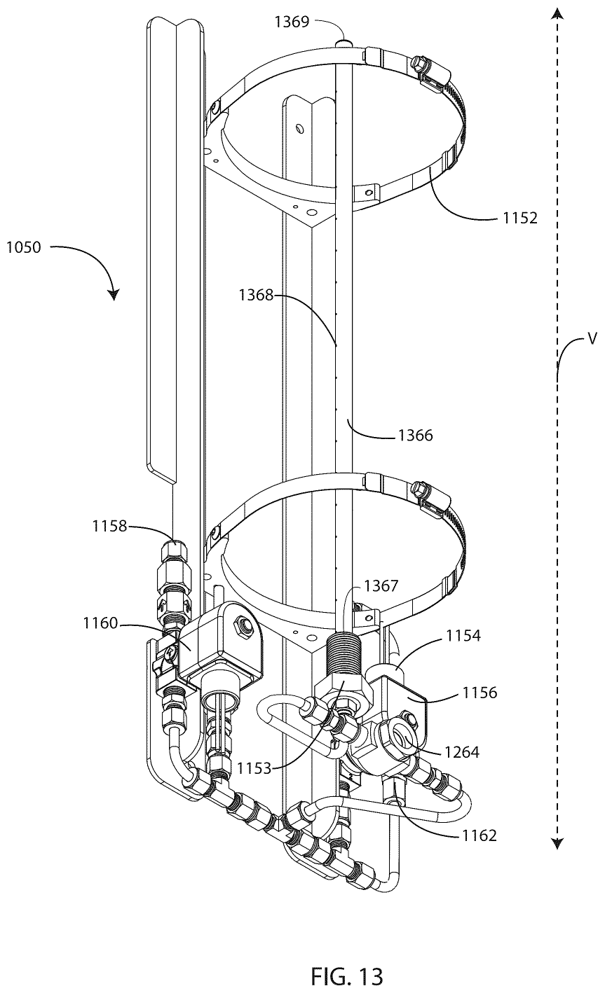

Referring now to FIG. 13, a perspective view of components of the accumulator is shown in accordance with embodiments herein. The accumulator 1050 depicted by FIG. 13 is identical to the accumulator 1050 depicted by FIG. 12 but with the storage tank 1152 removed for clarity.

In some embodiments, the refrigerant contained within the storage tank 1152 of the accumulator 1050 does not have a uniform composition. For instance, the refrigerant contained within the accumulator 1050 may separate. This phenomena is exacerbated when the refrigerant is a mixed refrigerant having components with different densities. In this scenario, the denser components of the mixed refrigerant tend to gravitate towards the bottom of the storage tank 1152. The separation of the refrigerant can cause issues when the refrigerant is drawn out of the storage tank 1152 via the tank opening 1153.

In scenarios where the accumulator 1050 is orientated such that the tank opening 1153 is disposed at the top of the storage tank 1152, a disproportionally small amount of the denser refrigerant components are released when the refrigerant is drawn out of the storage tank 1152 via the tank opening. In scenarios where the accumulator 1050 is orientated such that the tank opening 1153 is disposed at the bottom of the storage tank 1152 (such as illustrated by FIG. 13), a disproportionally large amount of the denser refrigerant components are released when the refrigerant is drawn out of the storage tank 1152 via the tank opening. Accordingly, the composition of the refrigerant (and consequently the fluid properties of the refrigerant) released form the storage tank 1152 will deviate from the expected composition, which may result in suboptimal performance of the closed-loop system for cryosurgery 100.

In various embodiments, the accumulator 1050 can incorporate one or more means to facilitate that the desired composition of refrigerant is consistently withdrawn from the storage tank 1152. In the example of FIG. 13, the accumulator 1050 can further include a straw 1366. In various embodiments, the straw 1366 can be mounted along a vertical axis V of the accumulator 1050. In some embodiments, the axis V is orientated in the direction of gravity. In various embodiments, the straw 1366 can be in fluid communication with the tank opening 1153 at its first end 1367 and can be sealed shut at its second end 1369. In various embodiments, the straw 1366 can have a length that spans approximately the depth of the storage tank 1152.

In various embodiments, the straw 1366 can contain a plurality of openings 1368 along its length. Accordingly, when is refrigerant is drawn out of the storage tank 1152 via the tank opening 1153, the refrigerant is sucked into the straw 1366 through each of the plurality of openings 1368. Consequently, rather than just being drawn out from a singular location of the storage tank 1152 through the tank opening 1153, the refrigerant is drawn out from multiple depths within the storage tank. In some embodiments, the straw 1366 facilitates a uniform drawing of fluid from all depths of the storage resulting in a uniform composition of refrigerant, even in scenarios when the refrigerant has become separated within the storage tank.

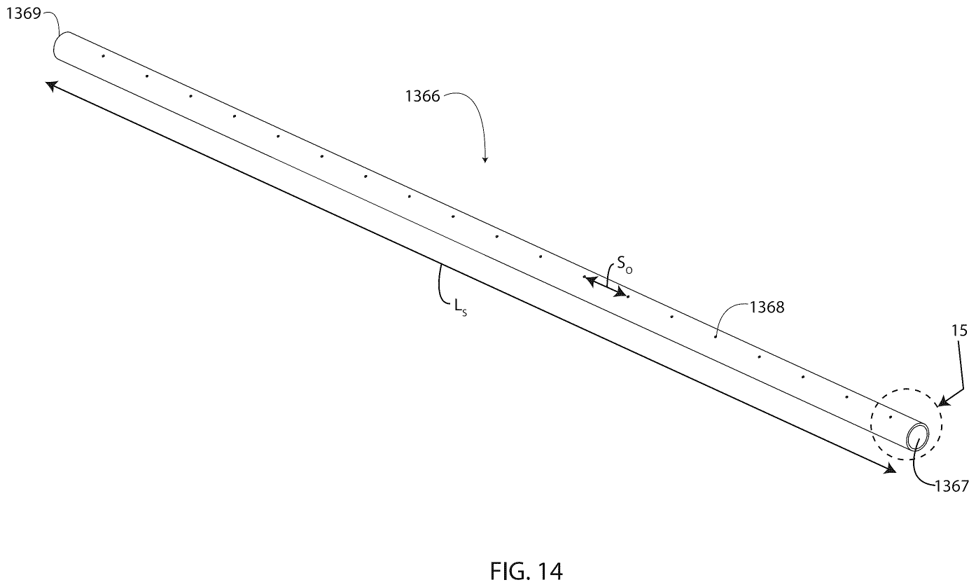

Referring now to FIG. 14, a schematic view of a straw is shown in accordance with embodiments herein. In various embodiments, the straw 1366 is open at its first end 1367 such that the straw can be in fluid communication with the tank opening 1153. In various embodiments, the straw 1366 is sealed shut at its second end 1369 such that the refrigerant is only drawn into the straw through the plurality of openings 1368 along its length L_{S}.

In various embodiments, the straw 1366 is constructed from any suitable material or material such as metals (e.g., stainless steel, copper, brass, or nickel alloys), polymers, (e.g., polymeric materials include polytetrafluoroethylene (PTFE), polyether ether ketone (PEEK), polyimide, polyethylene, polypropylene), or the like. In various embodiments, the plurality of openings 1368 are formed within the straw 1366 by any suitable processes, such as drilling, or the like.

In various embodiments, the straw 1366 can have a length L_{S}. In various embodiments, the length L_{S} is approximately equal to the depth of the storage tank 1152. In some embodiments, the length L_{S} can be greater than or equal to 30 cm, 40 cm, 50 cm, or 60 cm. In some embodiments, the length L_{S} can be less than or equal to 120 cm, 100 cm, 80 cm, or 60 cm. In some embodiments, the length L_{S} can fall within a range of 30 cm to 120 cm, or 40 cm to 100 cm, or 50 cm to 80 cm, or can be about 60 cm.

In various embodiments, the straw 1366 can contain any suitable number of openings 1368 along its length L_{S}. In some embodiments, the number of openings 1368 can be greater than or equal to 5, 10, 15, or 20 openings. In some embodiments, the number of openings 1368 can be less than or equal to 50, 40, 30, or 20 openings. In some embodiments, the number of openings 1368 can fall within a range of 5 to 50 openings, or 10 to 40 openings, or 15 to 30 openings, or can be about 20 openings.

In the example of FIG. 14, the straw contains a first set of 19 openings along the length L_{S} of the straw 1366. In various embodiments, the straw 1366 can contain multiple sets of openings. For instance, the straw depicted by FIG. 14, can have a second set of 19 (or any suitable number of openings) along the length L_{S} of the straw 1366 on the opposite side of the straw from the first set of openings.

In some embodiments, the openings 1368 can be uniformly distributed along the length L_{S} of the straw 1366. In some embodiments, the openings 1368 are not uniformly distributed along the length L_{S} of the straw 1366. For instance, in some embodiments, the openings 1368 may be spaced closer together towards the second end 1369 of the straw 1366 than at the first end 1367 of the straw (or vice versa). In various embodiments, each opening 1368 is spaced apart from the next along the length L_{S} of the straw 1366 by a spacing S_{O}. In various embodiments, the spacing S_{O} between the openings 1368 is selected as a function of the length L_{S} of the straw 1366 and/or the number of openings 1368 along the length L_{S}. In some embodiments, the spacing S_{O} can be greater than or equal to 5 mm, 12 mm, 18 mm, or 25 mm. In some embodiments, the spacing S_{O} can be less than or equal to 50 mm, 42 mm, 33 mm, or 25 mm. In some embodiments, the spacing S_{O} can fall within a range of 5 mm to 50 mm, or 12 mm to 42 mm, or 18 mm to 33 mm, or can be about 25 mm.

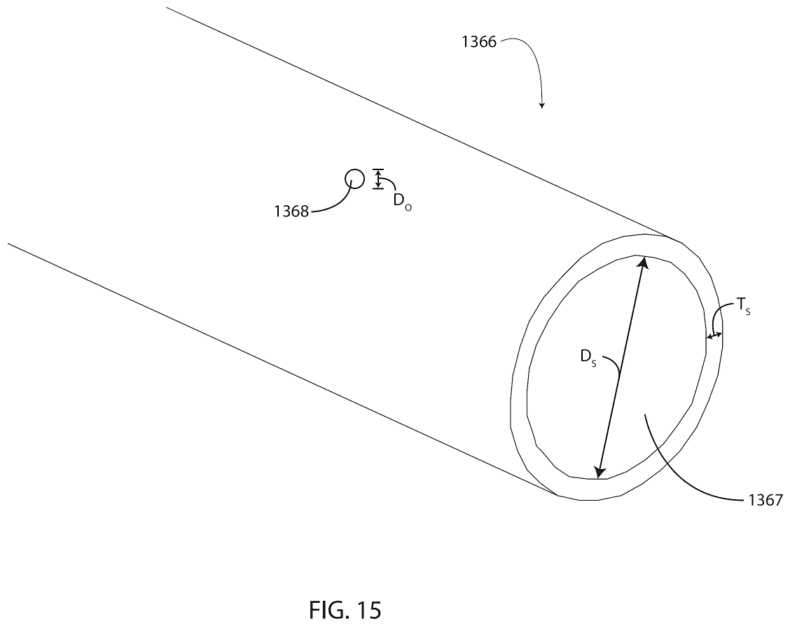

Referring now to FIG. 15, a detailed view of the straw of FIG. 14 about feature 15 is shown in accordance with embodiments herein. In various embodiments, the straw 1366 is open at its first end 1367 such that the straw can be in fluid communication with the tank opening 1153.

In various embodiments, the straw 1366 has a diameter D_{S} corresponding to an open area of the straw. In various embodiments, the straw 1366 is open at its first end 1367 and along its entire length until it is sealed closed at its second end 1369. In some embodiments, the diameter D_{S} is selected such that the cross-sectional area of the opening of the straw at the first end 1367 is approximately equal to the sum of the cross-sectional areas of each of the openings 1368 along the length of the straw. In some embodiments, the diameter D_{S} can be greater than or equal to 5 mm, 7 mm, 8 mm, or 10 mm. In some embodiments, the diameter D_{S} can be less than or equal to 30 mm, 23 mm, 17 mm, or 10 mm. In some embodiments, the diameter D_{S} can fall within a range of 5 mm to 30 mm, or 7 mm to 23 mm, or 8 mm to 17 mm, or can be about 10 mm.

In various embodiments, the straw 1366 has a wall thickness Tₛ. In various embodiments, the thickness Tₛ is selected to be sufficiently thick such that the straw remains rigid as the refrigerant is drawn in through the plurality of openings 1368. In some embodiments, the wall thickness Ts can be greater than or equal to 0.25 mm, 0.5 mm, 0.75 mm, or 1 mm. In some embodiments, the wall thickness Ts can be less than or equal to 2.50 mm, 2 mm, 1.50 mm, or 1.00 mm. In some embodiments, the wall thickness Ts can fall within a range of 0.25 mm to 2.50 mm, or 0.5 mm to 2.0 mm, or 0.75 mm to 1.5 mm, or can be about 1 mm.

In various embodiments each opening 1368 has a diameter D₀. In some embodiments, the diameter D_{O} can be greater than or equal to 0.25 mm, 0.5 mm, 0.75 mm, or 1 mm. In some embodiments, the diameter D_{O} can be less than or equal to 2.50 mm, 2 mm, 1.50 mm, or 1.00 mm. In some embodiments, the diameter D_{O} can fall within a range of 0.25 mm to 2.50 mm, or 0.5 mm to 2.0 mm, or 0.75 mm to 1.5 mm, or can be about 1 mm.

In some embodiments, all of the openings 1368 along the length of the straw 1366 have the same diameter. In some embodiments, the openings 1368 along the length of the straw 1366 can vary in diameter. For instance, in some embodiments, the openings 1368 have larger diameters towards the second end 1369 of the straw 1366 than at the first end 1367 of the straw 1366 (or vice versa).

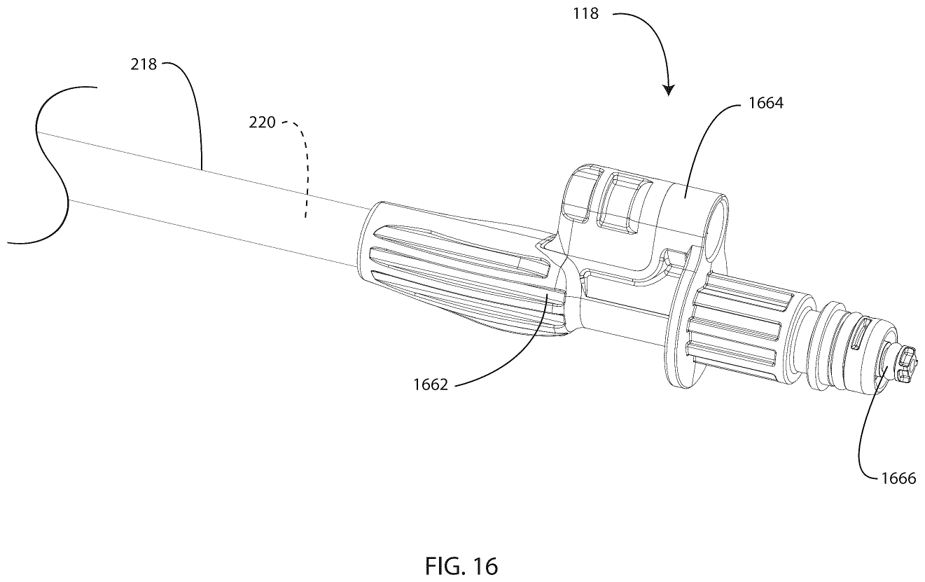

### Probe-Console Connector (FIG. 16)

Referring now to FIG. 16, a perspective, schematic view of a probe-console connector is shown in accordance with embodiments herein. In various embodiments, the probe-console connector 118 is configured to connect a cryoablation probe 101 to the cryoablation probe port 121 of the console 117 of the closed-loop system for cryosurgery 100.

In various embodiments, the probe-console connector 118 can include a main body 1662. The main body 1662 can be configured to sealingly connect to both the supply tube 220 and the return tube 218 of the cryoablation probe 101 to the console 117. In various embodiments, the main body 1662 the probe-console connector 118 is configured to provide a leak-tight seal between the cryoablation probe 101 and to the console 117, such that substantially none of the working fluid escapes from the closed-loop working fluid circuit as the working fluid circulates between the console 117 and the cryoablation probe 101.

In various embodiments, the probe-console connector 118 can further include an electrical connector 1664 configured to connect one or more electrical components to the cryoablation probe 101. Examples of electrical components for use with the cryoablation probe 101 can include connections for a vacuum circuit, a heater or cooler (e.g., electrical heater 480) and components for an imaging system. In various embodiments, the probe-console connector 118 can further include a console interface 1666 configured to connect with a mating interface feature within the console 117.

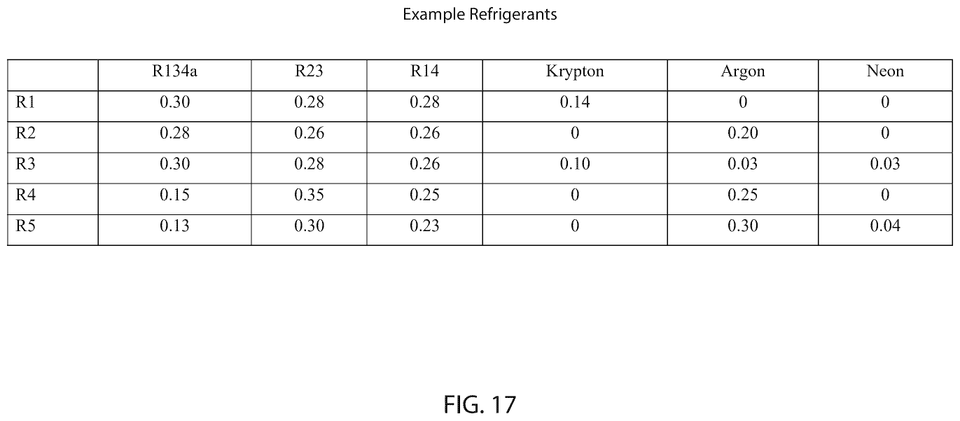

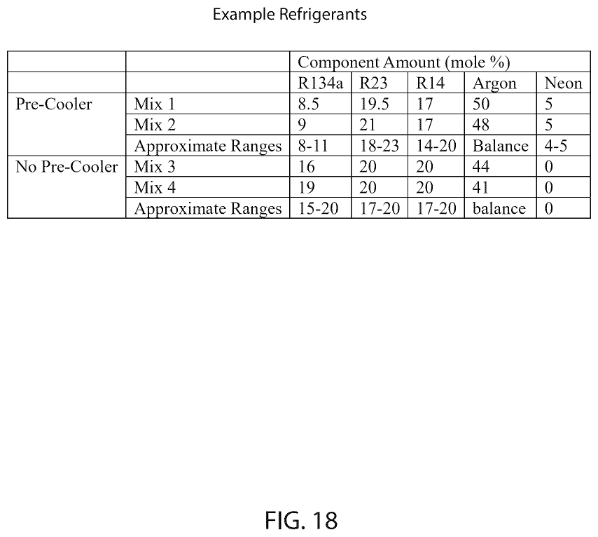

### Working Fluid (FIGS. 17-18)

To increase the feasibility utilizing a closed-loop working fluid circuit in a cryoablation system, such as the closed-loop system for cryosurgery depicted by FIGS. 8-10, it is desirable to utilize a working fluid that can undergo a Joule-Thomson transition at relatively low pressures. Single component working fluids, such as argon are compressed to relatively high pressures (>10 MPa) for a cryoablation procedure to be performed. Such working fluids provide cooling power while expanding from a very high pressure to a near ambient pressure. The high-pressure ratio required to perform a cryoablation procedure with such cooling fluids makes it energy intensive to recompress the low-pressure working fluid after expansion. As such, the low-pressure working fluid is generally released to the environment and the high-pressure working fluid is supplied from a pressure vessel, forming an open working fluid circuit.

In various embodiments, the working fluid comprises a mixed refrigerant. It has been found that certain mixed-refrigerants can undergo a Joule-Thomson transition at relatively low pressures (<5 MPa). Due to the lower pressure ratio, the mixed refrigerant working fluid can be recompressed relatively efficiently. Accordingly, the mixed refrigerant working fluid can be repeatedly recompressed and recirculated in a closed-loop system for cryosurgery.

In various embodiments, the mixed refrigerant comprises at least one of krypton and argon. In various embodiments, the composition of the mixed refrigerant can be configured to maximize the performance of the cryoablation system. In various embodiments, the mixed refrigerant is configured to attain cryogenic temperatures and provide sufficient cooling power to form an ice ball and ablate patient tissue.

In various embodiments, the mixed refrigerant consists of non-flammable and non-toxic components. For general applications, mixed refrigerants tend to be composed of a low boiling point, pure substance (e.g., nitrogen or argon) and one or more hydrocarbons. However, due to operator and patient safety concerns, it is desirable for a mixed refrigerant for use in a closed-loop system for cryosurgery to be devoid of any flammable components. Accordingly, hydrocarbons are not included in the mixed refrigerant for various embodiments of the disclosed closed-loop working fluid circuit. Examples of non-flammable and non-toxic refrigerant components can include R134a, R218, R23, R14 Xenon, Krypton, Argon, Nitrogen, and Neon.

In various embodiments, the molar concentration of R134a in a mixed refrigerant can be between about 0.0 and 0.35. In various embodiments, the molar concentration of R218 in a mixed refrigerant can be between about 0.0 and 0.35. In various embodiments, the molar concentration of R23 in a mixed refrigerant can be between about 0.0 and 0.60. In various embodiments, the molar concentration of R14 in a mixed refrigerant can be between about 0.0 and 0.50. In various embodiments, the molar concentration of Xenon in a mixed refrigerant can be between about 0.0 and 0.30. In various embodiments, the molar concentration of Argon in a mixed refrigerant can be between about 0.0 and 0.50. In various embodiments, the molar concentration of Nitrogen in a mixed refrigerant can be between about 0.0 and 0.50. In various embodiments, the molar concentration of Neon in a mixed refrigerant can be between about 0.0 and 0.15.

The refrigerants R134a, R218, R23, and R14 are available from The Chemours Company, having business offices in Wilmington, Delaware. Referring now to FIG. 17, a table of exemplary mixed refrigerants shown in accordance with embodiments herein. FIG. 17 shows five exemplary mixed refrigerants (R1-R5) suitable for use in the closed-loop system from cryoablation and the respective molar concentrations.

Referring now to FIG. 18, a table of exemplary mixed refrigerants is shown in accordance with embodiments herein. FIG. 18 shows four exemplary mixed refrigerants (Mix 1, Mix 2, Mix 3, Mix 4) suitable for use in the closed-loop system from cryoablation and the respective molar percentages of each component.

In various embodiments, the composition of the mixed refrigerant can vary depending on whether or not the closed-loop system for cryosurgery 100 contains a pre-cooler. In the example of FIG. 18, Mix 1 and Mix 2 are suitable for use in a closed-loop cryoablation system with a pre-cooler while Mix 3 and Mix 4 are suitable for use in a closed-loop cryoablation system without a pre-cooler.

In various examples, a mixed refrigerant suitable for use in closed-loop cryoablation system with a pre-cooler contains a molar percentage of between 8% and 11% of R134a, a molar percentage of between 18% and 23% of R23, a molar percentage of between 14% and 20% of R14, a molar percentage of between 4% and 5% of Neon, and the remaining molar percentage is balanced with Argon, with a molar percentage of between 41% and 66% of Argon.

In various examples, a mixed refrigerant suitable for use in closed-loop cryoablation system without a pre-cooler contains a molar percentage of between 15% and 20% of R134a, a molar percentage of between 17% and 20% of R23, a molar percentage of between 17% and 20% of R14, a molar percentage of 0% of Neon, and the remaining molar percentage is balanced with Argon, such as with a molar percentage of between 40% and 51% of Argon.

In various embodiments, the mixed refrigerant fluid is a zeotropic mixture. A zeotropic mixture as defined herein is a type of refrigerant mixture composed of two or more substances that have different boiling points. Unlike azeotropic mixtures, which behave like a single substance during phase change, individual substances within a zeotropic mixture do not evaporate or condense at the same temperature as one substance. As a result, zeotropic mixtures exhibit significant temperature changes during the phase transition between liquid and vapor.

When the working fluid is a zeotropic mixture, challenges can arise in keeping the various components of the zeotropic mixture mixed. In particular, as described in the context of FIGS. 8-9, as the working fluid cools, it transitions from a vapor to a mixed and liquid vapor phase. In various embodiments, the closed-loop system for cryosurgery 100 can include one or more measures for preventing the mixed phase working fluid from separating.

In an embodiment, certain components of the closed-loop system for cryosurgery can be placed at differing elevations from other components in order to minimize the separation of the working fluid. For instance, in some embodiments, lines and components of the closed-loop system for cryosurgery from the compressor 802 to the cryoablation probe 101 are configured to be arranged such that the working fluid flows downward toward the cryoablation probe 101. Such a configuration may promote mixing and minimize the liquid phase working fluid trapped at any specific location in the system. In an embodiment, rather than using pressure regulators, the closed-loop system for cryosurgery 100 can utilize an accumulator 1050 to regulate the pressure of the working fluid. In an embodiment, the closed-loop system for cryosurgery 100 can maintain the working fluid at temperatures and pressures that do not allow the working fluid to saturate.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A closed-loop system for cryosurgery, comprising:
a closed-loop working fluid circuit configured to contain a working fluid;
a compressor configured to increase a pressure of the working fluid;
a first heat exchanger downstream from the compressor, wherein the first heat exchanger is configured to lower a temperature of the working fluid;
a cryoablation probe, the cryoablation probe comprising:
a shaft comprising:
a supply tube; and
a return tube surrounding the supply tube;
an insulated zone;
an expansion chamber extending distally from the insulated zone, a cryoablation probe interface, the cryoablation probe interface comprising:
working fluid inlet; and
working fluid outlet;
wherein the system is configured so that, after exiting the first heat exchanger, the working fluid will:
enter the cryoablation probe via the working fluid inlet;
travel to the expansion chamber via the supply tube;
expand in the expansion chamber at a Joule-Thomson orifice, wherein upon expanding at the Joule Thompson orifice, the working fluid cools to a temperature of less than 150 Kelvin;
travel to the cryoablation probe interface via the return tube;
exit the cryoablation probe via the working fluid outlet;
enter the compressor downstream from the working fluid outlet; and
enter the first heat exchanger downstream from the compressor.

2. The closed-loop system of claim 1, wherein the working fluid comprises a mixed refrigerant.

3. The closed-loop system of claim 2, wherein the mixed refrigerant consists of non-flammable and non-toxic components.

4. The closed-loop system of claim 2, wherein the mixed refrigerant comprises at least one of krypton and argon.

5. The closed-loop system of claim 1, wherein the working fluid comprises a zeotropic mixture.

6. The closed-loop system of claim 1, further comprising an accumulator configured to store at least a portion of the working fluid.

7. The closed-loop system of claim 6, wherein the accumulator is configured to maintain a working pressure of the working fluid.

8. The closed-loop system of claim 1, further comprising a vacuum, wherein the vacuum is configured to pull the working fluid out from the cryoablation probe.

9. The closed-loop system of claim 1, further comprising a pre-cooler between the first heat exchanger and the working fluid inlet, wherein the pre-cooler is configured to further cool the working gas to a sub-ambient temperature.

10. The closed-loop system of claim 1, further comprising a vacuum circuit, wherein the vacuum circuit is defined within the insulated zone between the return tube and an insulating shaft.

11. The closed-loop system of claim 1, wherein the compressor is configured to increase the pressure of the working fluid to at least 3 MPa.

12. The closed-loop system of claim 1, wherein the system is configured so that the working fluid experiences a temperature drop of between about 120 Kelvin and 150 Kelvin within the cryoablation probe.

13. A method of operating a closed-loop system for cryosurgery comprising:
increasing a pressure of a working fluid in a closed-loop working fluid circuit with a compressor;
after increasing the pressure of the working fluid, lowering a temperature of the working fluid with a first heat exchanger;
after lowering the temperature of the working fluid, supplying the working fluid to a cryoablation probe via a working fluid inlet; the cryoablation probe comprising:
a shaft comprising:
a supply tube; and
a return tube surrounding the supply tube;
an insulated zone; and
an expansion chamber extending distally from the insulated zone;
sending the working fluid to the expansion chamber via the supply tube;
expanding the working fluid in the expansion chamber at a Joule-Thomson orifice, wherein upon expanding at the Joule Thompson orifice, the working fluid cools to a temperature of less than 150 Kelvin;
expelling the expanded working fluid from the cryoablation probe via the return tube and a working fluid outlet; and
returning the working fluid to the compressor.

14. The method of claim 13, wherein the working fluid comprises a mixed refrigerant.

15. The method of claim 14, wherein the mixed refrigerant consists of non-flammable and non-toxic components.

16. The method of claim 13, wherein the working fluid comprises a zeotropic mixture.

17. The method of claim 13, further comprising storing at least a portion of the working fluid in an accumulator.

18. The method of claim 17, wherein the accumulator is configured to maintain a working pressure of the working fluid.

19. The method of claim 13, wherein the compressor is configured to increase the pressure of the working fluid to at least 3 MPa.

20. The method of claim 13, wherein the system is configured so that the working fluid experiences a temperature drop of between about 10 Kelvin and 40 Kelvin when the working fluid expands at the Joule-Thomson orifice.
